(19) United States
(12) Patent Application Publication
(10) Pub. No.: US 2006/0173346 A1
(43) Pub. Date: Aug. 3, 2006

(54) Ultrasound diagnostic system and method for automatically activating a probe

(75) Inventor: Tae Ho Lee, Seoul (KR)

Correspondence Address: OBLON, SPIVAK, MCCLELLAND, MAIER & NEUSTADT, P.C., 1940 DUKE STREET, ALEXANDRIA, VA, 22314, US

(73) Assignee: Medison Co., Ltd., Hongchun-gun (KR)

(21) Appl. No.: 11/204,107

(22) Filed: Aug. 16, 2005

(30) Foreign Application Priority Data
Dec. 29, 2004 (KR) 10-2004-0114616

(51) Int. Cl. A61B 8/14; A61B 8/00

(52) U.S. Cl. 600/459; 600/437

(57) ABSTRACT

The present invention relates to an ultrasound diagnostic system and a method of automatically activating a probe. The ultrasound diagnostic system comprises: a probe; a sensor mounted on the probe for sensing a user touching the probe and generating a selection signal in response thereto; and a central controller for controlling an activation of the probe based on the selection signal.

### FIELD OF THE INVENTION

The present invention generally relates to an ultrasound diagnostic system, and more particularly to an ultrasound diagnostic system and a method for automatically activating a probe.

### BACKGROUND OF THE INVENTION

An ultrasound diagnostic system generally transmits ultrasound signals to organs and acquires ultrasound images by detecting reflected ultrasound signals from the organs. Particularly, the ultrasound diagnostic system is used to detect foreign materials within a living body, to measure a degree of lesion and to observe, for example, tumors and fetuses.

A conventional ultrasound diagnostic system comprises a plurality of probes, which transmit ultrasound signals to organs and receive ultrasound echo signals from the organs. It also comprises a user input unit and a display unit. In the conventional ultrasound diagnostic system, when a user selects a particular probe to use among various probes through the user input unit, a central controller within the conventional ultrasound diagnostic system controls an operation of the probe selected by the user. However, the conventional ultrasound system has a problem in that whenever the user selects a probe among various probes, the user must manually input selection information of the probe through the user input unit.

### SUMMARY OF THE INVENTION

It is an objective of the present invention to provide an ultrasound diagnostic system and a method for automatically activating a probe selected by a user through a sensor mounted on the probe.

In accordance with an aspect of the present invention, there is provided an ultrasound diagnostic system comprising: a probe; a sensor mounted on the probe for sensing a user touching the probe and generating a selection signal in response thereto; and a central controller for controlling an activation of the probe based on the selection signal.

In accordance with another aspect of the present invention, there is provided an ultrasound diagnostic system comprising: a plurality of probes; a sensor mounted on each of the plurality of probes for sensing a user touching a probe and generating a selection signal in response thereto; a central controller for controlling an activation of the probe based on the selection signal; a plurality of relays connected to each of the plurality of probes; and a relay controller for controlling each of the plurality of relays according to the control of the central controller.

In accordance with yet another aspect of the present invention, there is provided a method of automatically activating a probe in an ultrasound diagnostic system including a probe, a sensor mounted on the probe and a central controller, comprising the steps of: a) at the sensor, sensing a user touching the probe and generating a selection signal in response thereto; and b) at the central controller, controlling an activation of the probe based on the selection signal.

In accordance with still yet another aspect of the present invention, there is provided a method of automatically activating a probe in an ultrasound diagnostic system including a plurality of probes, a sensor mounted on each of the plurality of probes, a plurality of relays connected to each of the plurality of probes and a relay controller, comprising the steps of: a) at the sensor, sensing a user touching a probe and generating a selection signal in response thereto; b) at the central controller, generating a control signal based on the selection signal; and c) at the relay controller, switching a relay corresponding to the selected probe into an on-state, and switching the others into an off-state.

### BRIEF DESCRIPTION OF THE DRAWINGS

The above and other objects and features of the present invention will become apparent from the following description of preferred embodiments given in conjunction with the accompanying drawings, in which:

FIG. 1 is a front view of an ultrasound diagnostic system in accordance with the present invention;

FIG. 2 is a schematic diagram showing a plurality of probes, a main board and a probe select assembly board provided within a body in the ultrasound diagnostic system shown in FIG. 1; and

FIG. 3 is a flowchart illustrating a process of automatically activating a probe in accordance with the present invention.

### DETAILED DESCRIPTION OF THE PRESENT INVENTION

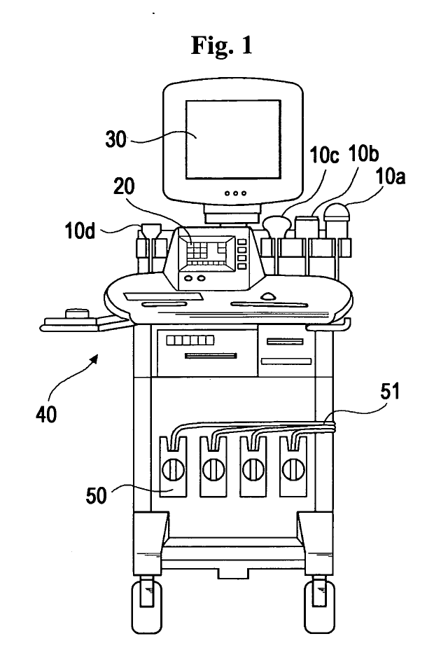

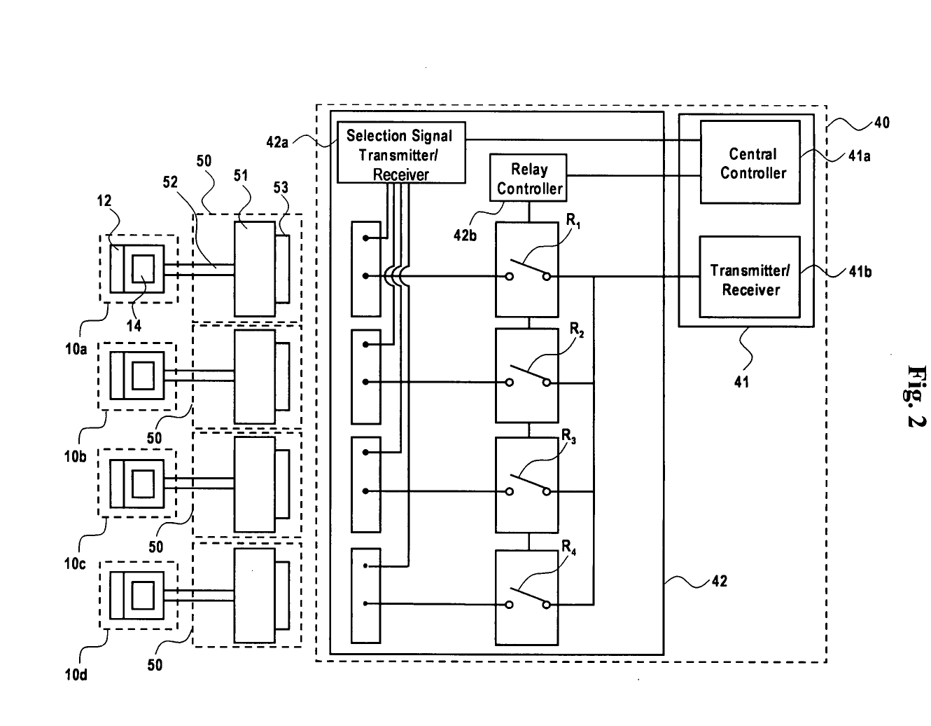

FIG. 1 is a front view of an ultrasound diagnostic system in accordance with the present invention, and FIG. 2 is a schematic diagram showing a plurality of probes, a main board and a probe select assembly board provided within a body in the ultrasound diagnostic system shown in FIG. 1.

Referring to FIGS. 1 and 2, the ultrasound diagnostic system in accordance with the present invention comprises: a plurality of probes 10a, 10b, 10c, and 10d, which transmit ultrasound signals to organs and receive ultrasound echo signals from the organs; a user input unit 20; a display unit 30; a body 40; and a connection unit 50 for connecting the probes 10a to 10d to body 40.

Each probe comprises a transducer 12 having a plurality of ultrasound elements and a sensor 14. The sensor 14, which is mounted on each probe 10a to 10d, senses a user touching a probe and generates a selection signal in response thereto. More particularly, when the user touches probe 10a among probes 10a to 10d, the sensor 14 mounted on the probe 10a generates a selection signal. Herein, it is desirable that the sensor 14 can detect the touch of the probe regardless of a position and aspect that the user touches a probe. In the embodiment, a capacitance sensor, a pressure sensor or a temperature sensor can be used as the sensor 14. Further, the sensor 14 may generate the selection signal when the user touches the probes 10a to 10d over a predetermined time, for example, a few seconds.

A body 40 comprises a main board 41 and a Probe Select Assembly (PSA) board 42. The main board 41 comprises a central controller 41a for controlling an activation of the probes and a transmitter/receiver 41b. The main board 41 further comprises a beam former (not shown), an image processor (not shown) and a scan converter (not shown). The PSA board 42 comprises: a selection signal transmitter/receiver 42a for receiving the selection signal generated from the sensor of the selected probe and transmitting the selection signal to the central controller 41a of the main board 41; relays R₁ to R₄ for connecting each probe 10a to 10d with transmitter/receiver 41b; and a relay controller 42b for controlling relays R₁ to R₄ based on a control signal transmitted from the central controller 41a. For example, when the user selects the probe 10a by various ways such as holding or touching the probe 10a, the selection signal transmitter/receiver 42a receives the selection signal from the sensor 14 mounted on the probe 10a and transmits the selection signal to the central controller 41a. The relay controller 42b activates the probe 10a by switching relay R₁ connected to the probe 10a into an on-state, while not activating the probes 10b to 10d by switching relays R₂ to R₄ connected to the probes 10b to 10d into an off-state.

In another embodiment of the present invention, the PSA board 42 may not comprise the selection signal transmitter/receiver 42a. In such a case, the selection signal transmitted from the sensor 14 is directly transmitted to the central controller 41a of the main board 41.

Each connection unit 50 comprises: a connector 51; a first connecting cable 52 for connecting the probe with the connector 51; and a second connecting cable 53 for connecting the connector 51 with the body 40. The connection unit 50 transmits data between the probes 10a to 10d and body 40. The connection unit 50 can be removed when data between the probes 10a to 10d and body 40 is transmitted via a wireless transmitter/receiver.

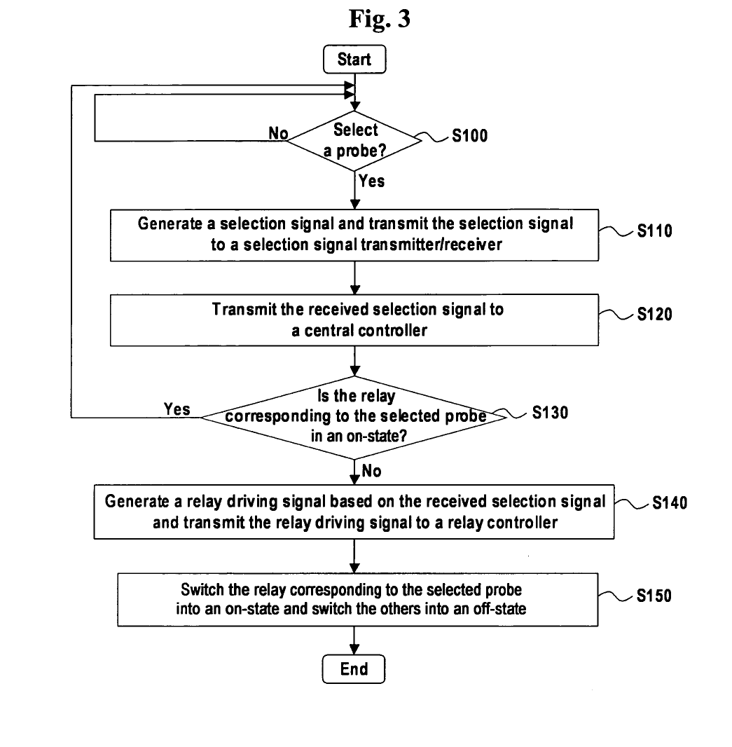

Hereinafter, a process for automatically activating a probe in accordance with the present invention will be described by reference of FIGS. 1-3.

The sensor 14 mounted on each probe 10a-10d senses whether a user selects a probe to use at step S100. If it is determined that a probe is not selected, then step S100 is performed. If it is determined that a probe is selected by the user, then the sensor 14 of the probe 10a generates a selection signal and transmits the selection signal to the selection signal transmitter/receiver 42a of the PSA board 42 at step S110.

The selection signal transmitter/receiver 42a transmits the selection signal to the central controller 41a of the main board 41 at step S120.

The central controller 41a determines whether a relay corresponding to the selected probe is in an on-state at step S130. If it is determined that the relay is in an on-state, then step S100 is performed to receive a selection of a probe. If it is determined that the relay is in an off-state, central controller 41a generates a relay driving signal which switches the relay corresponding to the selected probe into an on-state, the others into an off-state, based on the received selection signal, and transmits the relay driving signal to the relay controller 42a at step S140.

The relay controller 42a switches the relay corresponding to the selected probe to on-state, switches the others to off-state, based on the relay driving signal transmitted from the central controller 41a at step S150.

In another embodiment, two or more sensors may generate the selection signals and transmit the selection signals to the central controller 41a during a predetermined time. In such a case, the central controller 41a may display an alarm message for informing that a plurality of probes are selected and wait to select one probe. The alarm message may be sound or voice message. In another embodiment, the central controller 41a, which receives the selection signals during a predetermined time, may decide to choose a selection signal among the selection signals based on predetermined superiority, and perform step S140 and step S150 based on the decided selection signal.

As mentioned above, the present invention provides an effect in which a probe can be automatically activated so that a user does not need to input information for selecting a probe through the user input unit. Therefore, convenience can be provided to the user.

While the present invention has been described and illustrated with respect to a preferred embodiment of the invention, it will be apparent to those skilled in the art that variations and modifications are possible without deviating from the broad principles and teachings of the present invention which should be limited solely by the scope of the claims appended hereto.

What is claimed is:

1. An ultrasound diagnostic system, comprising:
a probe;
a sensor mounted on the probe for sensing a user touching the probe and generating a selection signal in response thereto; and
a central controller for controlling an activation of the probe based on the selection signal.

2. The ultrasound diagnostic system as recited in claim 1, wherein the sensor is a capacitance sensor, a pressure sensor or a temperature sensor.

3. The ultrasound diagnostic system as recited in claim 1, wherein the ultrasound diagnostic system further comprises a selection signal transmitting/receiving means for receiving the selection signal from the sensor and transmitting the selection signal to the central controller.

4. An ultrasound diagnostic system, comprising:
a plurality of probes;
a sensor mounted on each of the plurality of probes for sensing a user touching a probe and generating a selection signal in response thereto;
a central controller for controlling an activation of the probe based on the selection signal;
a plurality of relays connected to each of the plurality of probes; and
a relay controller for controlling each of the plurality of relays according to the control of the central controller.

5. The ultrasound diagnostic system as recited in claim 4, wherein the sensor is a capacitance sensor, a pressure sensor or a temperature sensor.

6. The ultrasound diagnostic system as recited in claim 4, wherein the ultrasound diagnostic system further comprises a selection signal transmitting/receiving means for receiving the selection signal from the sensor and transmitting the selection signal to the central controller.

7. A method of automatically activating a probe in an ultrasound diagnostic system including a probe, a sensor mounted on the probe, and a central controller, comprising the steps of:
a) at the sensor, sensing a user touching the probe and generating a selection signal in response thereto; and
b) at the central controller, controlling an activation of the probe based on the selection signal.

8. A method of automatically activating a probe in an ultrasound diagnostic system including a plurality of probes, a sensor mounted on each of the plurality of probes, a plurality of relays connected to each of the plurality of probes, and a relay controller, comprising the steps of:
a) at the sensor, sensing a user touching a probe and generating a selection signal in response thereto;
b) at the central controller, generating a control signal based on the selection signal; and
c) at the relay controller, switching a relay corresponding to the selected probe to on-state, and switching the others to off-state.
